(19) United States
(12) Patent Application Publication
(10) Pub. No.: US 2020/0116514 A1
(43) Pub. Date: Apr. 16, 2020

(54) A METHOD AND SYSTEM FOR ACQUIRING ATMOSPHERIC POLLUTION DATA AND REPORTING IT TO THE DRIVER OF A MOTORCYCLE

(71) Applicant: PIAGGIO & C. S.P.A., Pontedera, Pisa (IT)

(72) Inventor: Mario Donato SANTUCCI, Pontedera, Pisa (IT)

(73) Assignee: PIAGGIO & C. S.P.A., Pontedera, Pisa (IT)

(21) Appl. No.: 16/482,200

(22) PCT Filed: Jan. 26, 2018
(86) PCT No.: PCT/IB2018/050483

§ 371 Date: Jul. 30, 2019

(30) Foreign Application Priority Data
Jan. 31, 2017 (IT) 102017000010069

(51) Int. Cl. G01C 21/34; B60W 50/14; G08G 1/0968; G01C 21/36; H04W 4/021; G01N 33/00

(52) U.S. Cl. CPC G01C 21/3461; B60W 50/14; G08G 1/096844; G01N 33/0075; G01C 21/3691; H04W 4/021; G01C 21/3415

(57) ABSTRACT

A method for acquiring atmospheric pollution data and reporting the data to a motorcycle driver includes acquiring, when the motorcycle is being driven, by a sensor installed on board the motorcycle first datum correlated with atmospheric pollution in a geographical area where the motorcycle is used and obtaining a first signal containing the first datum. The first signal is provided to an electronic processing unit and processed by the electronic processing unit to generate a control signal. A signaling device is controlled by the control signal to provide the motorcyclist with information correlated with exposure of the motorcyclist to the atmospheric pollution when the motorcycle is driven. The processing step includes comparing the first datum with a threshold value for verifying whether the pollution acquired by the sensor exceeds a pre-set level. The signaling device is switched on if the pollution acquired exceeds the pre-set level.

The present disclosure relates to the technical field of motorcycles and in particular, it concerns a method and system for acquiring atmospheric pollution data and reporting it to the driver of a motorcycle.

As is known, growing levels of pollution in urban areas represent a serious risk, not only to pedestrians and cyclists, but also to drivers and passengers of motor vehicles themselves. In relation to this last category, the problem is particularly felt by drivers and passengers of motorcycles, as it is not possible to limit exposure to the outside atmosphere on board such vehicles, which are generally without a cubicle. At present, few solutions exist for such problem, other than those arising from steps undertaken by the competent authorities with laws relating to the control of emissions and measures for restricting traffic in certain periods of time and/or in certain urban areas.

One currently known method, which can be used by motorcyclists in an attempt to reduce the above problem may be to wear special masks containing filters. Such masks require particular hygiene, appropriate replacement and, in certain cases, particularly by some people, they may be considered irritating to wear.

It is a general object of the present description to provide a method and system, which allow the risk of motorcyclists' exposure to pollution agents to be reduced.

This and other objects are achieved by means of a method as defined in claim 1 in its more general embodiment and in its dependent claims in some particular embodiments.

The invention will be better understood from the following detailed description of its embodiments, given by way of example and which are not, therefore, limiting in any way in relation to the appended drawings, wherein:

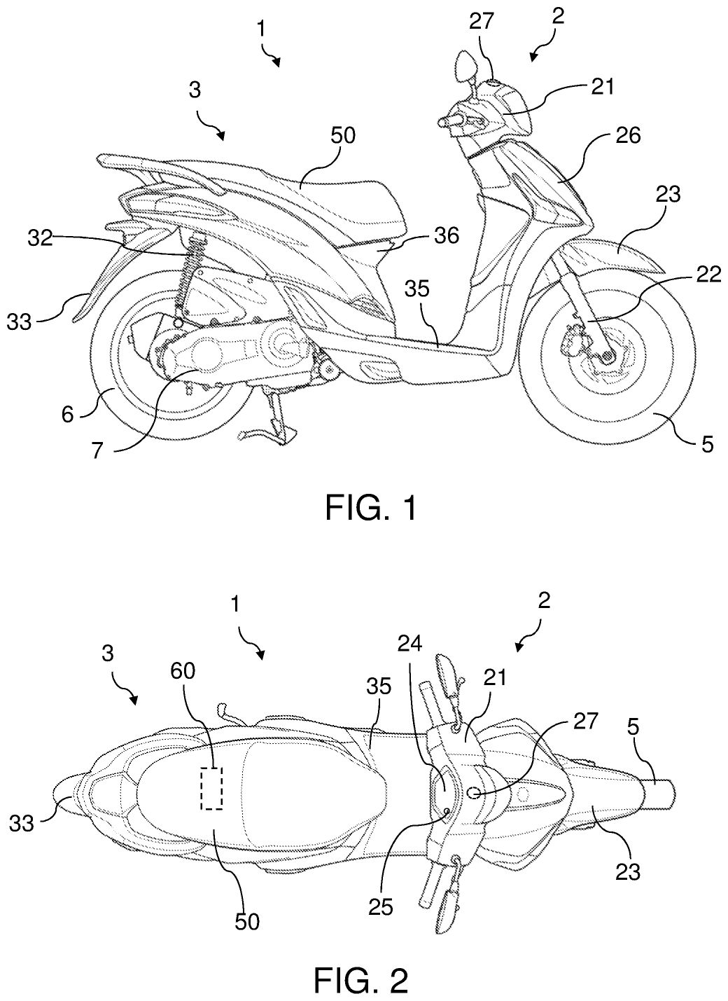

FIG. 1 shows a schematic side view of a non-limiting embodiment of a motorcycle comprising an atmospheric pollution sensor;

FIG. 2 shows a schematic plan view of the motorcycle in FIG. 1;

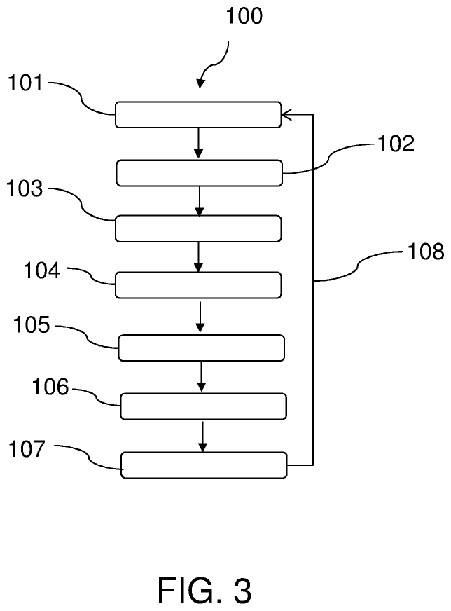

FIG. 3 shows a schematic flow chart of a method for acquiring atmospheric pollution data and reporting it to the driver of a motorcycle.

Equal or similar elements have been indicated with the same numerical references in the appended figures.

The figures show an embodiment of a motorcycle 1, which, in the particular illustrated example, is conceived without including any limitation, as a motorcycle 1 with two wheels, and in particular as a scooter with two wheels, having a forecarriage wheel 5 and a back wheel 6.

From now on in the present description, reference will be made to a general motorcycle 1, meaning that the following description can generally be applied to any type of motor vehicle 1 in category L comprising:
a motorcycle body 2,3;
at least two wheels 5, 6 constrained to the motorcycle body 2,3;
a propulsion engine 7, for example thermal or electric or hybrid, constrained to the motorcycle body 2,3 and operatively connected to at least one of the two wheels 5,6.

For example, the aforesaid motorcycle 1 is a motorcycle with two wheels, such as, for example, a scooter or a motorcycle, or a tricycle with three wheels, of which at least two steering and tilting front wheels, or a quadricycle with two pairs of tilting wheels of which at least two steering.

The motorcycle body 2, 3 has a forecarriage 2 and a rearcarriage 3.

In the example, the forecarriage 2 comprises steering handlebars 21, a fork 22, the forecarriage wheel 5, a front fender 23, an instrument panel 24.

In the example, the rearcarriage 3 comprises a saddle 50, one or two rear suspensions 32, the rear wheel 6, the propulsion engine 7, a rear fender 33, a footboard 35, an under saddle support 36.

The motorcycle 1 also comprises at least one sensor 27 adapted to acquire, for example adapted to measure, at least one first datum correlated with the atmospheric pollution, for example correlated with the concentration in the atmosphere of at least one polluting agent, such as, for example, carbon monoxide and/or carbon dioxide and/or fine dust and/or nitric oxide and/or ethanol. The sensor 27 is, for example a gas sensor.

The sensor 27 is adapted to supply an output electric signal containing the aforesaid first datum acquired.

Preferably, as shown in the illustrated example, the sensor 27 is installed at the forecarriage 2 of the motorcycle 1, for example on the steering handlebars 21. In alternative embodiments, the sensor 27 is mounted onto a front windshield of the motorcycle 1 or onto the front shield 26 of the body of the motorcycle 1. In any case, it is advantageous to mount the aforesaid sensor 27 onto a front portion of the motorcycle 1 body 2, 3.

The sensor 27 is operatively connected to an electronic processing unit 60, which is inside or outside the motorcycle 1. For example, the sensor 27 is connected to the electronic processing unit 60 by means of one or more electric wires, or by means of at least one wireless communication channel, for example, a Bluetooth channel.

According to a first embodiment, for example, the electronic processing unit 60 is comprised, in other words installed on board the motorcycle 1 and it is a dedicated unit or a unit also adapted to perform other operations related to the working of the motorcycle 1 itself. For example, the electronic processing unit is the ECU (Engine Control Unit) of the motorcycle 1 or an independent processing unit, operatively connected to the ECU. In the particular example illustrated in FIG. 2, the electronic processing unit 60 (not visible from the outside and consequently drawn with a dotted line) is placed in housing below the saddle 50 of the motorcycle 1, for example inside or below a space arranged below the saddle 50 of the motorcycle 1.

In a second embodiment, the aforesaid electronic processing unit 60 is outside the motorcycle 1, or rather not installed on the motorcycle 1, and it is a mobile device, for example, for personal communication, such as, for example a smartphone or a tablet-pc or a smartwatch, which the driver can carry or wear when he/she is on board the motorcycle 1. In this case, the motorcycle 1 is fitted with a communication interface, for example, a Wi-Fi interface, or a USB or Bluetooth interface adapted to connect the sensor 27 to the external electronic processing unit.

The first and the second embodiment described above can be separate from each other, but also joined. For example, it is possible to include a hybrid or a dual embodiment, wherein part of the processing is carried out by a first electronic processing unit inside the motorcycle 1 and part of the processing is carried out by a second electronic processing unit outside the motorcycle 1, wherein the first and second processing unit are operatively connected to each other.

The electronic processing unit 60 is operatively connected to at least one signaling device 25, for example an optical and/or acoustic and/or touch signaling device. In the particular illustrated example, the signaling device is for example an optical signaling device (graphic and/or luminous) comprised in the control panel 24 of the motorcycle 1, for example a display mounted on board. In an alternative or complementary embodiment, the signaling device is a display of an external mobile device for personal communication of the type described above (smartphone, smartwatch, etc.) and/or a vibrating element of said external device.

In further embodiments, the aforesaid signaling device could also be, or include an acoustic signaling device, such as, for example, a buzzer or a loudspeaker installed on the motorcycle 1 and/or on the external personal communication mobile device.

According to one embodiment, the electronic processing unit 60 is operatively connected to, or comprises, a satellite localization device adapted to record the geographical coordinates of the motorcycle 1, for example, in a GPS geolocalization system.

According to one embodiment, the electronic processing unit 60 comprises a communication interface adapted to connect said processing unit 60 to a remote processing station, for transmitting and/or receiving data correlated with atmospheric pollution.

Some embodiments of a method 100 for acquiring atmospheric pollution data and reporting it to the driver of a motorcycle will be described in detail starting from the aforesaid structural and functional characteristics of the motorcycle 1 and the relative acquisition and signaling system. A simplified and non-limiting flow chart of such method 100 is illustrated in FIG. 3.

According to a general embodiment, the method 100 comprises the following steps:
acquiring 101, while the motorcycle 1 is being driven, by means of a sensor 27, installed on board the motorcycle 1, as said previously, at least one first datum correlated with the atmospheric pollution in a geographical area in which the motorcycle 1 is localized and obtaining at least one first signal containing said first datum;
providingproviding 102 the first signal to an electronic processing unit 60, as described previously;
processing 106 the first signal by means of the electronic processing unit 60 in order to generate a control signal;
controlling 107 a signaling device 25, as described previously, by means of the control signal, in order to provide the motorcyclist of the motorcycle 1 with information correlated with the exposure of the motorcyclist to the atmospheric pollution when the motorcycle 1 is being driven.

The aforesaid steps of the method 100 are carried out preferably, and without limitation, in sequence, also in a non-strictly consecutive manner, in the sense that one or more additional interposed steps can be included between two successive steps.

The processing step 106 comprises an operation of comparing the first datum with at least one threshold value, to verify whether the pollution detected, that is to say acquired, by the sensor 27 is greater than a pre-set level. In the controlling step 107 the signaling device 25 is turned on if the pollution detected exceeds the pre-set level. For example, the controlling step 107 can be such as to bring the signaling device 25 from an off state to an on state (for example, switching on an orange or a red light, or activating an icon on an LCD display) should it emerge from the operation of comparing that the level of pollution detected is greater than the pre-set level, and that it is such as to keep the signaling device in such state until the detected pollution becomes less than the pre-set level. In the operation of comparing, comparisons with several threshold values are preferably included, for example, for reporting various different levels of pollution of increasing intensity to the driver, according to a step of preferred fine discretization.

The method 100 can be based exclusively on local pollution measurements, in other words measurements performed on board the motorcycle 1, even if it is useful and advantageous to integrate such measurements with pollution data provided from other sources, for example, other vehicles (not necessarily only motorcycles) or fixed pollution recording centers located along the itinerary when the motorcycle 1 is being driven. For this reason, according to an advantageous embodiment, the method 100 also comprises a step of transmitting 103 the first datum to a remote processing station designed to collect and aggregate pollution data coming from various sources, such as those described above. Preferably, at least one geographical localization datum of the motorcycle 1 is also transmitted during the transmission step 103, for example the coordinates of the motorcycle 1 in a GPS geolocalization system.

Thanks to the data transmitted in real time from the various sources, the remote processing station can provide the motorcycle 1 with additional data, for example information on the average level of pollution of an area in which the motorcycle 1 is used. For this reason, according to an advantageous embodiment, the method 100 also comprises the steps of:
obtaining or calculating 104, at the remote processing station, a second datum correlated with the average pollution of an area in which the motorcycle 1 is used when it is being driven;
sending 105 a second signal containing the second datum from the remote processing station to the electronic processing unit 60;
   and wherein the second signal is also processed by the electronic processing unit 60 during the processing step 106.

For example, the processing step 106 comprises an operation of comparing said first datum with said second datum, for example, in order to report to the driver that the particular place in which he/she finds himself/herself (for example, if the motorcycle 1 is located behind a particularly polluting vehicle, at a standstill or traveling behind such polluting vehicle) presents levels of pollution greater than an average pollution level obtained from pollution measurements relating to the place itself or to a position provided from various sources, substantially relating to the same time period.

In this way, on the basis of the signals provided by the signaling device 25, the driver of the motorcycle 1 can decide whether to move slightly, for example distancing himself/herself from a polluting vehicle by overtaking it, or falling back, or changing lane or he/she can even decide to change course, if the problem is not attributable to one vehicle in particular. To this end, in one embodiment, the processing step 106 also comprises the operations of:
calculating a navigation itinerary of the motorcycle 1 going from a starting location to a selected destination location;
correlating the navigation itinerary with atmospheric pollution data detected on sections of said itinerary and associating with said itinerary a first atmospheric pollution index on said itinerary;
identifying one or more alternative itineraries in order to find an alternative itinerary, if one exists, with which a second atmospheric pollution index on the alternative itinerary is associated, which is less than the first index.

In the aforesaid embodiment, the processing step 106 preferably comprises an operation of proposing the alternative itinerary found to the motorcyclist and preferably also an operation of receiving an acceptance or refusal datum of the alternative itinerary found from the motorcyclist. These operations can be carried out, for example, by means of an input and visualization interface, such as a display touch screen comprised in the control panel 24 or in an external mobile device for personal communication (e.g.: smartphone, tablet-pc, smartwatch).

For the purposes of calculating the alternative navigation itineraries, the pollution data is, for example that supplied to the remote processing station from the various sources described above, consequently, in this step there is interaction between the electronic processing unit 60 and the remote processing station.

As indicated by the arrow 108 in FIG. 3 the steps of the method 100 described above can be carried out iteratively and with continuity according to several cycles of acquisition, processing and signaling, starting from the start-up of the motorcycle 1, for example from the ignition of the panel 24 until the switching off of the same.

On the basis of the above explanation, it is therefore possible to understand how a method 100 of the type described above allows the previously stated objects to be achieved with reference to the state of the prior art.

Note that the general description set out above for the method 100 also corresponds to the description of a system for acquiring atmospheric pollution data and reporting it to the driver of a motorcycle 1, comprising:

a motorcycle 1 comprising a sensor 27, installed on board the motorcycle 1, adapted and configured to acquire at least one first datum correlated with the atmospheric pollution in a geographical area in which the motorcycle 1 is used when the motorcycle 1 is being driven, to obtain at least one first signal containing said first datum;

an electronic processing unit 60 adapted and configured to receive the first signal and process said first signal by means of 60 in order to generate a control signal;

a signaling device 25 adapted and configured to be controlled by means of the control signal for being switched-on if the pollution detected from the sensor (27) exceeds the pre-set level in order to provide the motorcyclist with information correlated with the exposure of the motorcyclist to the atmospheric pollution when the motorcycle 1 is being driven.

Finally, note that the general description set out above for the method 100 also corresponds to the description of a computer program comprising a program code, which can be loaded into a memory of an electronic processor for executing the steps of the method 100 described above, in its general embodiment, or preferred and/or advantageous embodiments.

Without prejudice to the principle of the invention, the embodiments and embodiment details can be varied widely with respect to the description and illustration given purely by way of a non-limiting example, without going beyond the scope of the invention, as defined in the appended claims.

1. A method for acquiring atmospheric pollution data and reporting the atmospheric pollution data to a driver of a motorcycle, comprising the steps of:
acquiring, when the motorcycle is being driven, by a sensor installed on board the motorcycle, at least one first datum correlated with atmospheric pollution in a geographical area in which the motorcycle is used during driving and obtaining at least one first signal containing said first datum;
providing the first signal to an electronic processing unit;
processing the first signal by the electronic processing unit to generate a control signal;
controlling a signaling device by the control signal to provide the driver with information correlated with exposure of the driver to the atmospheric pollution when the motorcycle is being driven;
wherein:
the processing step comprises an operation of comparing the first datum with at least one threshold value for verifying whether the pollution acquired by the sensor exceeds a pre-set level;
in the controlling step, the signalling device is switched-on if the pollution acquired exceeds the pre-set level.

2. A method according to claim 1, wherein the signaling device is an optical signaling device and/or acoustic signaling device and/or tactile signaling device.

3. A method according to claim 1, comprising a step of transmitting the first datum to a remote processing station to collect and aggregate pollution data coming from a plurality of sources.

4. A method according to claim 3, wherein at least one geographical localization datum of the motorcycle is transmitted during the transmission step.

5. A method according to claim 4, comprising the steps of:
obtaining or calculating, at the remote processing station, a second datum correlated with average pollution of an area in which the motorcycle is used when the motorcycle is being driven;
sending a second signal containing the second datum, from the remote processing station to the electronic processing unit;
wherein the second signal is processed during the processing step.

6. A method according to claim 5, wherein the processing step comprises an operation of comparing said first datum with said second datum.

7. A method according to claim 1, wherein the processing step comprises the operations of:
calculating a navigation itinerary of the motorcycle, going from a starting location to a selected destination location;
correlating said navigation itinerary with atmospheric pollution data detected on sections of said itinerary and associating with said itinerary a first atmospheric pollution index on said itinerary;
identifying one or more alternative itineraries to find an alternative itinerary, if an alternative itinerary exists, with which a second atmospheric pollution index on the alternative itinerary is associated, which is less than the first index.

8. A method according to claim 7, wherein the processing step comprises an operation of proposing the alternative itinerary found to the driver.

9. A method according to claim 8, wherein the processing step comprises a step of receiving an acceptance or refusal datum of the alternative itinerary found from the driver.

10. A computer program comprising a program code, the program code being loadable into a memory of an electronic processor for executing a method according to claim 1.

11. A system for acquiring atmospheric pollution data and reporting the atmospheric pollution data to a driver of a motorcycle, comprising:
a motorcycle comprising a sensor installed on board the motorcycle adapted and configured to acquire at least one first datum correlated with atmospheric pollution in a geographical area in which the motorcycle is used when the motorcycle is being driven to obtain at least one first signal containing said first datum;
an electronic processing unit adapted and configured to receive the first signal and process said first signal to generate a control signal;
a signaling device adapted and configured to be controlled by the control signal for being switched on if the pollution detected the sensor exceeds a pre-set level, to provide the driver with information correlated with exposure of the driver to the atmospheric pollution when the motorcycle is being driven.
